(19) United States
(12) Patent Application Publication
(10) Pub. No.: US 2022/0380841 A1
(43) Pub. Date: Dec. 1, 2022

(54) Methods and Kits using Internal Standards to Control for Complexity of Next Generation Sequencing(NGS) Libraries

(71) Applicant: The University of Toledo, Toledo, OH (US)

(72) Inventors: James C. Willey, Toledo, OH (US); Erin L. Crawford, Toledo, OH (US)

(73) Assignee: The University of Toledo, Toledo, OH (US)

(21) Appl. No.: 17/775,932

(22) PCT Filed: Nov. 13, 2020
(86) PCT No.: PCT/US20/60333

§ 371 Date: May 11, 2022

Related U.S. Application Data
(60) Provisional application No. 62/935,705, filed on Nov. 15, 2019.

(51) Int. Cl. C12Q 1/6869

(52) U.S. Cl. CPC C12Q 1/6869; C12Q 2600/166; C12Q 2600/156

(57) ABSTRACT

Described herein are kits and methods for quantifying the amount of at least one nucleic acid of interest in a sample. The kits include a mixture of synthetic spike-in internal standards (IS) reagents, where the spike-in IS reagents comprise a complexity calibration ladder (CCL) that includes synthetic internal standard competitors for at least one target gene.

### CROSS-REFERENCE TO RELATED APPLICATIONS

This application claims the benefit of U.S. provisional application Ser. No. 62/935,705 filed Nov. 15, 2019, the entire disclosures of which are expressly incorporated herein by reference.

### STATEMENT REGARDING FEDERALLY SPONSORED RESEARCH

This invention was made with government support under Grant Number CA086368 awarded by the National Institutes of Health and Early Detection Research Network Sub-Award 0000921356 awarded by the National Cancer Institute. The government has certain rights in the invention.

### FIELD OF THE INVENTION

The present invention relates methods for standardized sequencing of nucleic acids and uses thereof.

### BACKGROUND

The identification of genetic information is becoming a key piece of information for the diagnosis and treatment of many diseases. In order to make such diagnostic tool readily available, it is desired that this identification be as efficient and as inexpensive as possible. For diagnostic, medical, regulatory and ethical aspects, this identification should be as accurate as possible in order to rule out false measurements.

In addition to the desire to acquire human genetic material information, there is great interest in acquiring genetic information on, for example, mitochondria, pathogens and organisms that cause diseases.

One method for acquiring information is the Sanger sequencing method of genome analysis. Other methods are becoming available which provide an improved performance when compared with the Sanger sequencing method. These methods include a short high density parallel sequencing technology, next generation sequencing (i.e., NextGen or “NGS”), which are attempting to provide a more comprehensive and accurate view of RNA in biological samples than the Sanger sequence method.

Next-generation sequencing (NGS) is useful in a multitude of clinical applications by virtue of its automated and highly parallelized analysis of nucleic acid templates. However, the limit of clinical questions that NGS can address is largely determined by: i) the upstream source of nucleic acid template (e.g., human tissue, microbial sample, etc.), and ii) whether the clinically relevant biological variation in the nucleic acid template is greater than the technical variation (which is often introduced by such variants as workflow for sample preparation, sequencing and/or data analysis).

The workflow for NGS library preparation varies widely, but can broadly be grouped into one of two approaches: 1) digestion or fragmentation of the nucleic acid sample with subsequent ligation to a universal adaptor sequence, or 2) PCR with target specific primers that incorporate a universal adaptor sequence at their 5′ ends. In both approaches, if a nucleic acid template is RNA, a reverse transcription step is used to create the requisite DNA template for sequencing.

One concern with NGS is that these quantitative sequencing methods have high intra-lab and inter-lab variation. This problem thus reduces the value of any results, and has prevented the use of these sequencing methods in molecular diagnostics.

For example, non-systematic (i.e., non-reproducible) biases (i.e., errors), are often inadvertently introduced during preparation of the sequencing library. These non-systemic biases are a major roadblock to implementing NGS as a reliable and efficient routine measurement of nucleic acid abundance (quantification) in the clinical setting.

The most likely source of non-systematic bias (thus preventing inter-laboratory comparison, and hence routine clinical use, of quantitative NGS data) stems from issues arising from nucleic acid fragmentation, adaptor ligation and PCR.

Also, although not explicitly required, the FDA has issued guidance and industry recommendations that PCR-based in vitro diagnostic (IVD) devices should contain internal amplification controls (IAC) to control for interfering substances and verify that a negative result for a sample is not caused by inhibitors.

In addition, in order to avoid stochastic sampling error and ensure reliable measurements, it is necessary to sequence (i.e., read) a sufficient number of copies of the analyte being measured. One problem is that the range of transcript representation following library preparation often remains very high, typically one million-fold or greater, imposing high cost. This is because the transcripts from each gene must be sequenced at least 10 times (ensure 10 “reads”). To ensure 10 reads for the least represented genes, it is necessary to read a gene represented at one million fold higher level at least 10 million times.

Recently, the use of Unique Molecular identifiers (UMI) for amplicon libraries and the use of random fragment end measurement in hybrid capture libraries have been used to determine library complexity in NGS. However, due to the amount of inherent sequencing error, the number of reads required to confidently determine uniqueness is significantly higher with UMI, therefore requiring more space on a sequencing flow cell and cost per sample. Additionally. both UMIs and random fragment end analysis are reported to have biases leading to a non-random distribution when measured altering interpretation of results and potentially skewing data

Thus, a NGS method that reduces inter-experimental and inter-laboratory variation in measurement of nucleic acid copy number in samples will be of great use to both research and clinical applications.

### SUMMARY OF THE INVENTION

In a first aspect, described herein is a kit for quantifying the amount of at least one nucleic acid of interest in a sample that includes spike-in internal standard (IS) reagents present as a complexity calibration ladder (CCL) that contain multiple synthetic internal standard (IS) sequences at different concentrations.

The IS sequence, at each concentration, contains a nucleotide change at a different position along the sequence so that each IS sequence can be distinguished from the IS sequence at each other concentration.

The multiple internal standard (IS) sequences are mixed at different known concentrations relative to each other, and at a known ratio to IS for other targets in an internal standard

In certain embodiments, wherein the spike-in IS reagents comprise one or more of:

i) an endogenous complexity calibration ladder (ECCL) that includes synthetic internal standard competitors for at least one endogenous target gene; and,

ii) an alien complexity calibration ladder (ACCL) that includes synthetic internal standard competitors for at least one alien target gene.

In certain embodiments, the internal standard (IS) sequences are used with one or more of: 1) PCR amplification, 2) ligation, 3) hybrid or other types of capture, 4) linear or other forms of amplification, and 5) sequencing.

Also described herein are a methods for measurement of the level of complexity of Next Generation Sequencing (NGS) libraries, comprising using the kits described herein 1.

Also described herein are a methods for control for technical variation in library preparations, comprising using the kits described herein.

Also described herein are a methods for estimate lower limit of detection for variant allele fraction, comprising using the kits described herein.

In certain embodiments, the endogenous target gene has PCR primers with known high efficiency, and a lack of reported pseudogenes.

In certain embodiments, the endogenous complexity calibration ladder (ECCL) is combined with an alien complexity calibration ladder (ACCL) that is not competitive with the at least one endogenous target gene and is not affected by a sample's biological properties.

In certain embodiments, the alien complexity calibration ladder (ACCL) comprises at least one of the External RNA Controls Consortium (ERCC) sequences.

In certain embodiments, after synthesis of the IS sequences (e.g., first/second/third/fourth/fifth/etc.) with nucleotide changes at different positions, multiple IS with different nucleotide changes are mixed at a different known concentrations relative to each other, and at a known ratio to other target IS in an internal standard mixture.

In another aspect, there is described herein methods for determining the complexity of a library preparation for each target in a specimen. The method includes comparing the reads of each other target IS to each IS in a complexity ladder to determine efficiency of library preparation for each target in each specimen, and, therefore, the number of molecules measured for each target in each specimen.

In certain embodiments, the complexity analysis includes the following steps:

1) identifying ECCL IS minD copies of ECCL IS loaded for which there are at least 5 reads (or some chosen minimum reads);

2) determining ECCL detection correction factor=ECCL IS minL/ECCL IS mind where ECCL IS minL is the number of copies loaded of the least concentrated IS in the ECCL;

3) calculating ECCL IS1 detected by multiplying IS1 copies loaded by the ECCL detection correction factor;

4) calculating ECCL NT copies detected using the formula [(ECCL NT reads/ECCL IS1 reads)*ECCL IS1 copies detected; and,

5) calculating detectable target NT copies using the formula [(target NT reads/ECCL NT reads)*ECCL NT copies detected*(target IS loaded/ECCL IS1 loaded).

In certain embodiments, the complexity analysis includes the following steps:

1) identifying ACCL IS mind copies of ACCL IS loaded for which there are at least 5 reads (or some chosen minimum reads);

2) determining ACCL detection correction factor=ACCL IS minL/ACCL IS mind where ACCL IS minL is the number of copies loaded of the least concentrated IS in the ACCL;

3) calculating ACCL IS1 detected by multiplying IS1 copies loaded by the ACCL detection correction factor;

4) calculating target IS copies detected by multiplying ACCL IS1 copies detected by target IS reads/ACCL IS1 read; and,

5) calculating target NT copies detected by multiplying target IS copies detected by target NT reads/target IS reads.

In certain embodiments, the kit comprising reagents for measurement of multiple low variant allele frequency (VAF) mutants in a target genes; and, instructions therefor.

In certain embodiments, the kit further includes reagents for measurement of expression and/or somatic mutations in multiple genes in a sample of cells. Such kit can include: PCR primers for each target gene, synthetic internal standard for each target gene, reagents to prepare PCR products as a library for next generation sequencing and/or oligonucleotide baits.

In certain embodiments, the variant allele frequency VAF<0.01%.

In certain embodiments, wherein the variant allele frequency VAF is about 5×10-4 (0.05%).

In certain embodiments, inclusion of the internal standards reliably measures mutations at a variant frequency as low as 0.05%, and 5% without the inclusion of the internal standards.

In certain embodiments, inclusion of the internal standards reliably measures mutations at a variant frequency as low as 0.05%.

In certain embodiments, the kit or method enables measurement of variant allele frequency VAF as low as 0.05% without any qualifications (e.g., 5% without inclusion).

In certain embodiments, use of the internal standards reliably measures low variant frequency mutations with VAF as low as 0.01% without use of unique molecular indices (UMI).

In certain embodiments, synthetic internal standards are included.

In certain embodiments, the method further comprises diagnosing whether a subject is at risk of developing a disease, comprising:

a) obtaining a biological sample from the subject;

b) measuring the levels of set of target genes in the biological sample using any one of the kits of any one of the claims herein so as to obtain physical data to determine whether the levels in the biological sample is higher than the levels in a control;

c) comparing the levels in the biological sample with the levels in the control;

d) distinguishing between true mutations and artifacts by controlling for sources of imprecision, false positives, and false negatives; and,

e) identifying the subject is at risk of developing the disease if the physical data indicate that the levels in the biological sample are significantly different from the levels in the control.

In certain embodiments, the method further comprises:

a) determining an actionable treatment recommendation for a subject diagnosed with a disease, comprising:

b) obtaining a biological sample from the subject detecting at least one feature that meets the threshold criteria for a positive value, using a set of probes that hybridize to and amplify a set of target genes to detect at least one feature with a positive value; and,

c) determining, based on the at least one positive feature with positive value detected, an actionable treatment recommendation for the subject.

In certain embodiments, the method further comprises:

determining a method of treatment for patients at risk of developing a disease wherein before medical management (e.g., screening for the disease and/or preventive treatment), risk of developing the disease is assessed by using any one of the kits as claimed herein; and:

the patients at low risk for developing the disease are subject to routine long term evaluation; and subsequently administering the medical treatment; and,

the patients at high risk of developing the disease or affected by the disease are subjected to screening for the disease, and/or medical treatment to prevent the disease, medical and/or radiation, and/or surgery.

In certain embodiments, measurement of low VAF mutants, comprises: calculation of limit of detection/limit of quantification for measurement of each analyte in each specimen, based on measurement of specimen analyte relative to a known number of synthetic internal standard molecules.

In certain embodiments, the method comprises conducting the following steps:

step 1) multiplex gradient PCR to enable primers with varying melting temperatures to anneal to specific target;

step 2) single-plex PCR followed by quantification and equimolar mixing enables equal loading onto sequencer; and.

step 3) PCR targets chosen based on high occurrence in the disease.

In certain embodiments, the diagnosis or evaluation comprises one or more of a diagnosis of a disease, a diagnosis of a stage of the disease, a diagnosis of a type or classification of the disease, a diagnosis or detection of a recurrence of the disease, a diagnosis or detection of a regression of the disease, a prognosis of the disease, or an evaluation of the response of a disease to a surgical or non-surgical therapy.

In certain embodiments, the test subject has undergone surgery for solid tumor resection and/or chemotherapy, and/or radiation treatment.

In certain embodiments, the method further comprises a step where the patients are subjected to ongoing short-term evaluation.

In certain embodiments, the method further comprises a step where the patients are subjected to therapy with therapeutic drugs.

In another aspect, described herein are uses of the kits and methods to facilitate approval by FDA and other regulatory agencies in kit form in regional laboratories.

In another aspect, described herein are uses of the kits and methods to measure mutations in cells that will then guide targeted therapies.

In another aspect, described herein are uses of the kits and methods to facilitate approval by FDA and other regulatory agencies of testing for measurement of mutations in the cells that will then guide targeted therapy of the disease in kit or method form in regional laboratories.

In another aspect, described herein are uses of the kits and methods to facilitate approval by FDA and other regulatory agencies of testing for measurement without unique molecular indices (UMI) of very low VAF (as low as 0.01%) mutations in the cells that will then guide targeted therapy of the disease in kit or method form in regional laboratories.

In another aspect, described herein are uses of the kits and methods to enable measurement of very low VAF mutations in the cells.

In another aspect, described herein are uses of the kits and methods to measure mutations in cells that will then guide targeted therapy of the disease.

In another aspect, described herein are uses of the kits and methods to measure mutations in a set of genes in normal cells to determine risk for the disease.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIG. 1. Schematic illustration of how to design internal standard (IS) spike-in molecules for NGS.

FIG. 2. Frequency of observed sequence variations for native template group and internal standards group for different types of sequence variations.

FIG. 3. Internal standard error for four replicates, showing the individual replicate error and mean error.

FIG. 4A. Hybrid capture panel for exons EGFR_18 (red), EGFR_20 (blue) and EGFR_21 (green), showing IS frequency (%).

FIG. 4B. NT frequency (%) showing replicate measurement, LOB, and variant allele frequency for exons EGFR_18 (red), EGFR_20 (blue) and EGFR_21 (green).

FIG. 4C. Comparison of expected, NT, reported NT and reported IS for exons EGFR_18 (red), EGFR_20 (blue) and EGFR_21 (green).

FIG. 5. Applying Internal Standards to fragmented FDA Samples.

FIG. 6. Transition Sequencing Error at TP53 (exon 6) Across 19 Internal Standard Replicates, showing the Variant Allele Frequency for TP53 transactivation domain, TP53 DNA binding domain, and TP53 tetramerization domain.

FIG. 7. TP53 (exon 6) Transition Variants in Sample 7.

FIG. 8. Mutations in 19 Patient Specimens Relative to IS.

FIG. 9. Example of an endogenous complexity calibration ladder (ECCL).

FIG. 10. Example of an alien complexity calibration ladder (ACCL).

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Definitions and Abbreviations

IS—Internal Standard, synthetic DNA

ISM—Internal Standard Mixture

NGS—Next Generation Sequencing

NT—Native Template, from targeted region of specimen DNA

PCR—Polymerase Chain Reaction

SNP—Single Nucleotide Polymorphism

VAF—Variant Allele Frequency

A “gene” is one or more sequence(s) of nucleotides in a genome that together encode one or more expressed molecules, e.g., an RNA, or polypeptide. The gene can include coding sequences that are transcribed into RNA which may then be translated into a polypeptide sequence, and can include associated structural or regulatory sequences that aid in replication or expression of the gene.

A “set” of markers, probes or primers refers to a collection or group of markers probes, primers, or the data derived therefrom, used for a common purpose (e.g., assessing an individual's risk of developing cancer). Frequently, data corresponding to the markers, probes or primers, or derived from their use, is stored in an electronic medium. While each of the members of a set possess utility with respect to the specified purpose, individual markers selected from the set as well as subsets including some, but not all of the markers, are also effective in achieving the specified purpose.

“Specimen” as used herein can refer to material collected for analysis, e.g., a swab of culture, a pinch of tissue, a biopsy extraction, a vial of a bodily fluid e.g., saliva, blood and/or urine, etc. that is taken for research, diagnostic or other purposes from any biological entity.

Specimen can also refer to amounts typically collected in biopsies, e.g., endoscopic biopsies (using brush and/or forceps), needle aspirate biopsies (including fine needle aspirate biopsies), as well as amounts provided in sorted cell populations (e.g., flow-sorted cell populations) and/or micro-dissected materials (e.g., laser captured micro-dissected tissues). For example, biopsies of suspected cancerous lesions, commonly are done by fine needle aspirate (FNA) biopsy, bone marrow is also obtained by biopsy, and tissues of the brain, developing embryo, and animal models may be obtained by laser captured micro-dissected samples.

“Biological entity” as used herein can refer to any entity capable of harboring a nucleic acid, including any species, e.g., a virus, a cell, a tissue, an in vitro culture, a plant, an animal, a subject participating in a clinical trial, and/or a subject being diagnosed or treated for a disease or condition.

“Sample” as used herein can refer to specimen material used for a given assay, reaction, run, trial and/or experiment. For example, a sample may comprise an aliquot of the specimen material collected, up to and including all of the specimen. As used herein the terms assay, reaction, run, trial and/or experiment can be used interchangeably

In some embodiments, the specimen collected may comprise less than about 100,000 cells, less than about 10,000 cells, less than about 5,000 cells, less than about 1,000 cells, less than about 500 cells, less than about 100 cells, less than about 50 cells, or less than about 10 cells.

In some embodiments, assessing, evaluating and/or measuring a nucleic acid can refer to providing a measure of the amount of a nucleic acid in a specimen and/or sample, e.g., to determine the level of expression of a gene. In some embodiments, providing a measure of an amount refers to detecting a presence or absence of the nucleic acid of interest. In some embodiments, providing a measure of an amount can refer to quantifying an amount of a nucleic acid can, e.g., providing a measure of concentration or degree of the amount of the nucleic acid present. In some embodiments, providing a measure of the amount of nucleic acid refer to enumerating the amount of the nucleic acid, e.g., indicating a number of molecules of the nucleic acid present in a sample. The “nucleic acid of interest” may be referred to as a “target” nucleic acid, and/or a “gene of interest,” e.g., a gene being evaluated, may be referred to as a target gene. The number of molecules of a nucleic acid can also be referred to as the number of copies of the nucleic acid found in a sample and/or specimen.

As used herein, “nucleic acid” can refer to a polymeric form of nucleotides and/or nucleotide-like molecules of any length. In certain embodiments, the nucleic acid can serve as a template for synthesis of a complementary nucleic acid, e.g., by base-complementary incorporation of nucleotide units. For example, a nucleic acid can comprise naturally occurring DNA, e.g., genomic DNA; RNA, e.g., mRNA, and/or can comprise a synthetic molecule, including but not limited to cDNA and recombinant molecules generated in any manner. For example the nucleic acid can be generated from chemical synthesis, reverse transcription, DNA replication or a combination of these generating methods. The linkage between the subunits can be provided by phosphates, phosphonates, phosphoramidates, phosphorothioates, or the like, or by nonphosphate groups, such as, but not limited to peptide-type linkages utilized in peptide nucleic acids (PNAs). The linking groups can be chiral or achiral. The polynucleotides can have any three-dimensional structure, encompassing single-stranded, double-stranded, and triple helical molecules that can be, e.g., DNA, RNA, or hybrid DNA/RNA molecules.

A nucleotide-like molecule can refer to a structural moiety that can act substantially like a nucleotide, for example exhibiting base complementarity with one or more of the bases that occur in DNA or RNA and/or being capable of base-complementary incorporation. The terms “polynucleotide,” “polynucleotide molecule,” “nucleic acid molecule,” “polynucleotide sequence” and “nucleic acid sequence,” can be used interchangeably with “nucleic acid” herein. In some specific embodiments, the nucleic acid to be measured may comprise a sequence corresponding to a specific gene.

In some embodiments the specimen collected comprises RNA to be measured, e.g., mRNA expressed in a tissue culture. In some embodiments the specimen collected comprises DNA to be measured, e.g., cDNA reverse transcribed from transcripts, and genomic DNA). Additionally, quality (sequence information) as well as quantity of nucleic acids can be assessed. Variant alleles and gDNA copy number also may be measured along with transcript abundance.

In some embodiments, the nucleic acid to be measured is provided in a heterogeneous mixture of other nucleic acid molecules.

The term “native template” as used herein can refer to nucleic acid obtained directly or indirectly from a specimen that can serve as a template for amplification. For example, it may refer to cDNA molecules, corresponding to a gene whose expression is to be measured, where the cDNA is amplified and quantified.

The term “primer” generally refers to a nucleic acid capable of acting as a point of initiation of synthesis along a complementary strand when conditions are suitable for synthesis of a primer extension product.

The term “library complexity” generally relates to the number of unique molecules in the “library” that is sampled by finite sequencing.

The term “Next Generation Sequencing (NGS) library complexity” relates to the number of unique starting target molecules in the sample or reaction, not limited to just those sequenced because other factors may influence whether a starting molecule is sequenced.

The method for controlling NGS library complexity includes the “spike-in” of a “complexity calibration ladder” of synthetic internal standard competitors (IS) for a target nucleic acid that is present in a clinical specimen. The term “spike-in” generally refers to a process which where something added to a sample or solution prior to further processing to fix the relationship between the thing spiked in and the other components of the sample or solution.

The term “complexity calibration ladder” refers to synthetic internal standard competitors (IS) for a target specimen. This target must have certain characteristics, including: PCR primers with known high efficiency and, lack of reported pseudogenes.

General Description

Described herein are kits and methods for assessing amounts of a nucleic acid in a sample. In some embodiments, the method allows measurement of small amounts of a nucleic acid, for example, where the nucleic acid is expressed in low amounts in a specimen, where small amounts of the nucleic acid remain intact and/or where small amounts of a specimen are provided.

Design of Internal Standard (IS) Spike-In Molecules for NGS

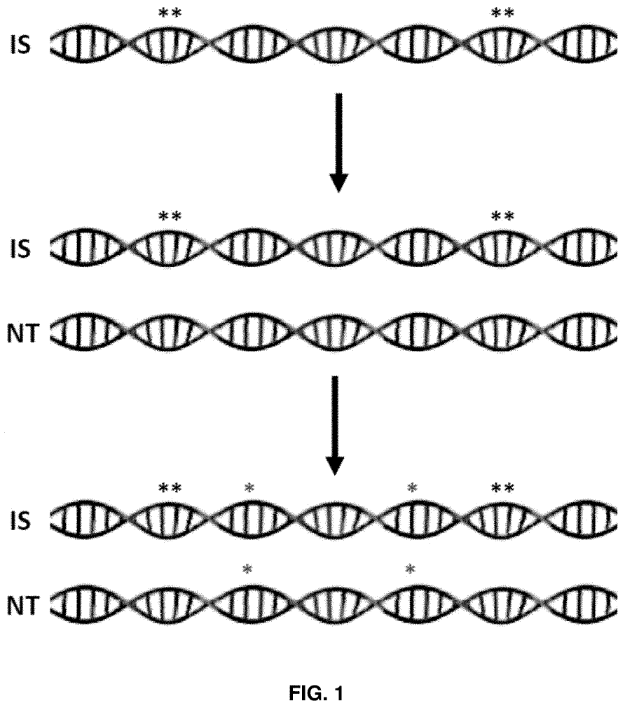

Referring first to FIG. 1, a schematic illustration of how to design internal standard (IS) spike-in molecules for NGS is shown.

It is to be understood that the use of methods described herein of using spike-in synthetic 1S to measure NGS library complexity with fewer reads per sample and with less bias, are compatible with the use of target specific IS, as described in the Willey et al. U.S. Pat. No. 9,944,973 (the entire disclosures of which are expressly incorporated herein by reference) which allows for improved measurement of sequencing error.

Internal Standards (IS) are synthetic DNA molecules that are homologous with a target nucleic acid, except for having known dinucleotide changes. That is, the IS behave the same as, but are distinguishable from target DNA native template (NT).

Use of IS allows for the ability to: 1) quantify measurable genome copies of each target analyte NT in library preparation; and 2) quantify and characterize nucleotide site-specific technical error.

In general, to prepare IS: 1) mix sample DNA with known number of IS molecules at 1:1 genome copy ratio prior to NGS library preparation; 2) co-amplify IS+NT mixture; 3) prepare sequencing library; and, 4) sequence sample.

Internal Standard “Spike-In Molecules” are custom perl script which separates IS reads from sample reads using dinucleotide changes. The error profile in native template (NT) nearly identical in internal standard (IS).

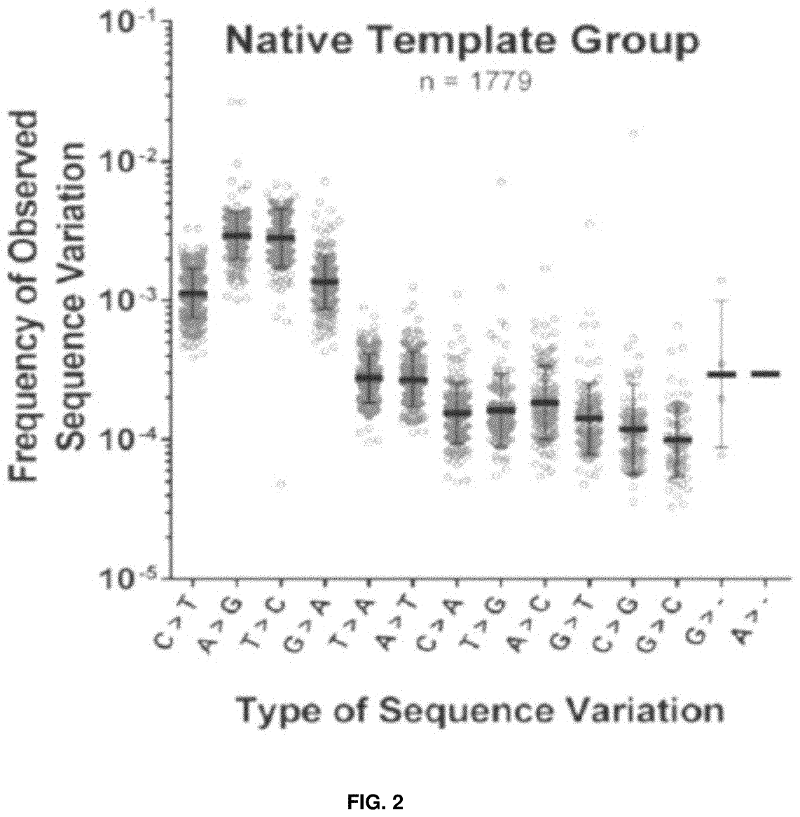

Thus, IS controls for library-specific error profiles, as shown in FIG. 2, which shows the frequency of observed sequence variations for native template group and internal standards group for different types of sequence variations.

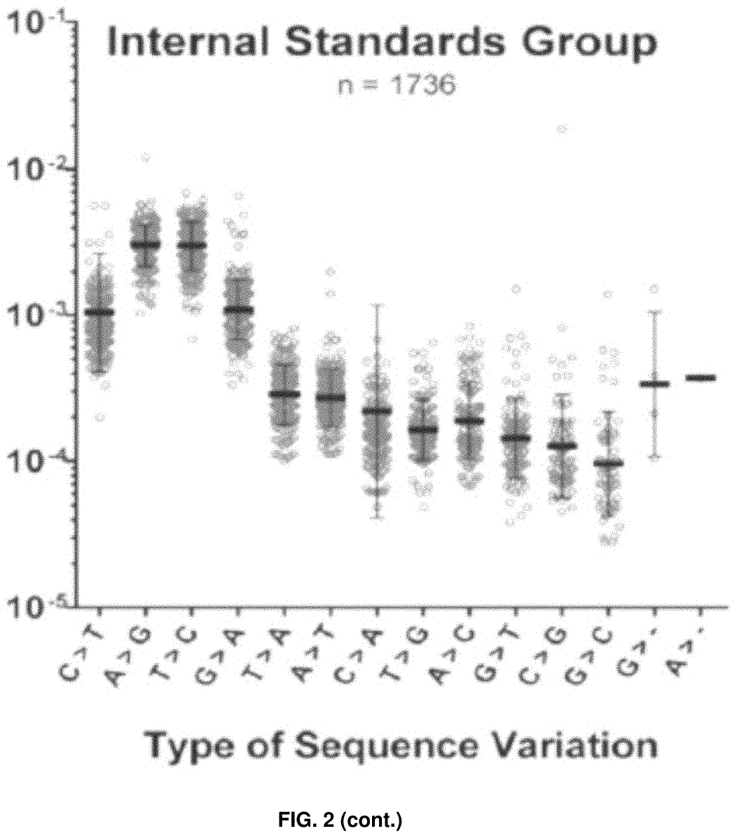

Additionally, as shown in FIG. 3, the nucleotide-specific technical error is reproducible. FIG. 3 shows the internal standard error for four replicates, showing the individual replicate error and mean error. The nucleotide-specific technical error at each NT base position matches corresponding IS position. Also, DNA landscape affects sequencing error on a region-to-region and nucleotide-to-nucleotide basis 4 IS and NT behave the same way.

Spiking IS into each reaction thus controls for variation within library preparation (e.g., interfering substances, intra- and inter-panel hybridization efficiency, ligation efficiency, amplification).

Internal standards also control for sources of imprecision enabling narrow confidence interval at each nucleotide: nucleotide-specific error frequency; platform-specific errors, and polymerase-specific errors.

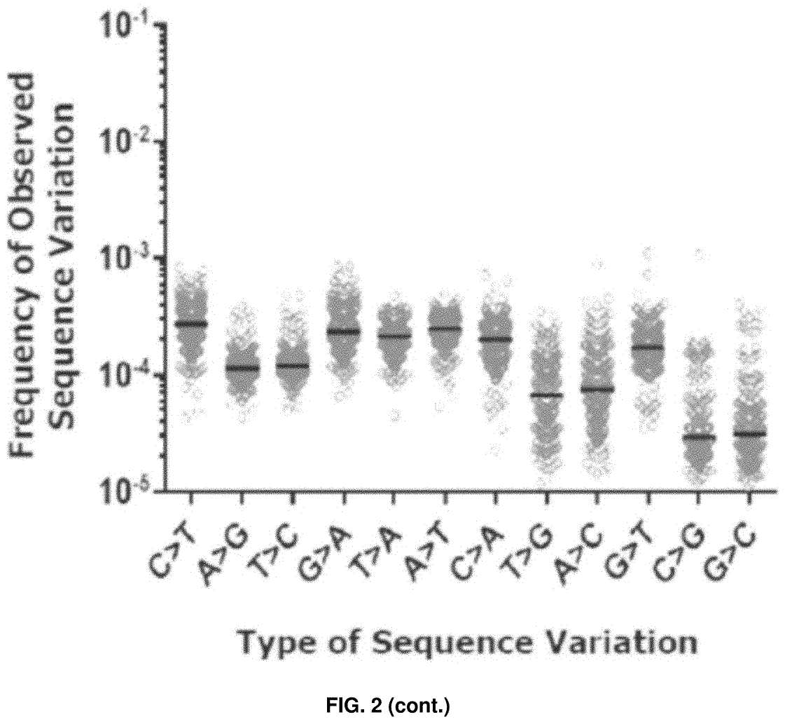

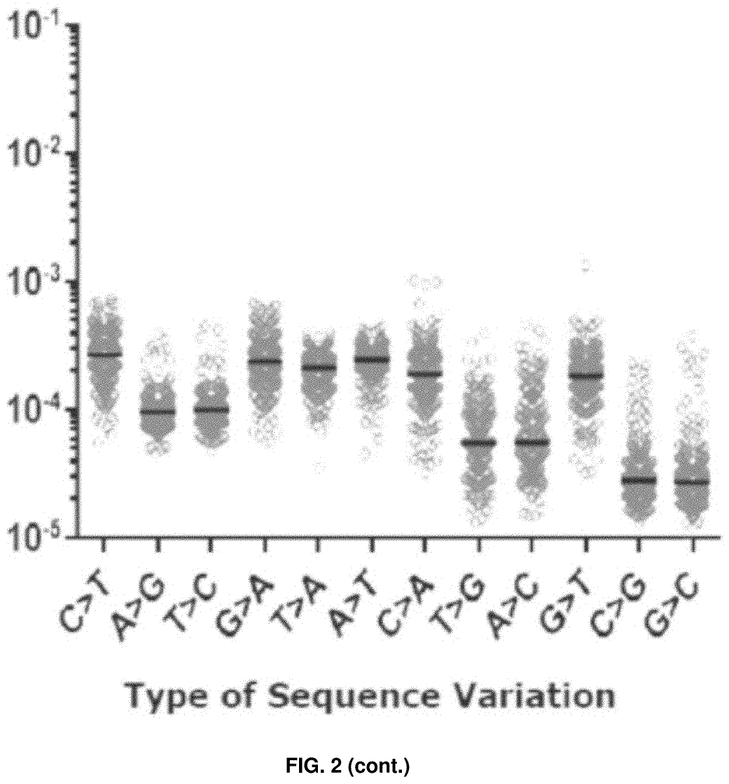

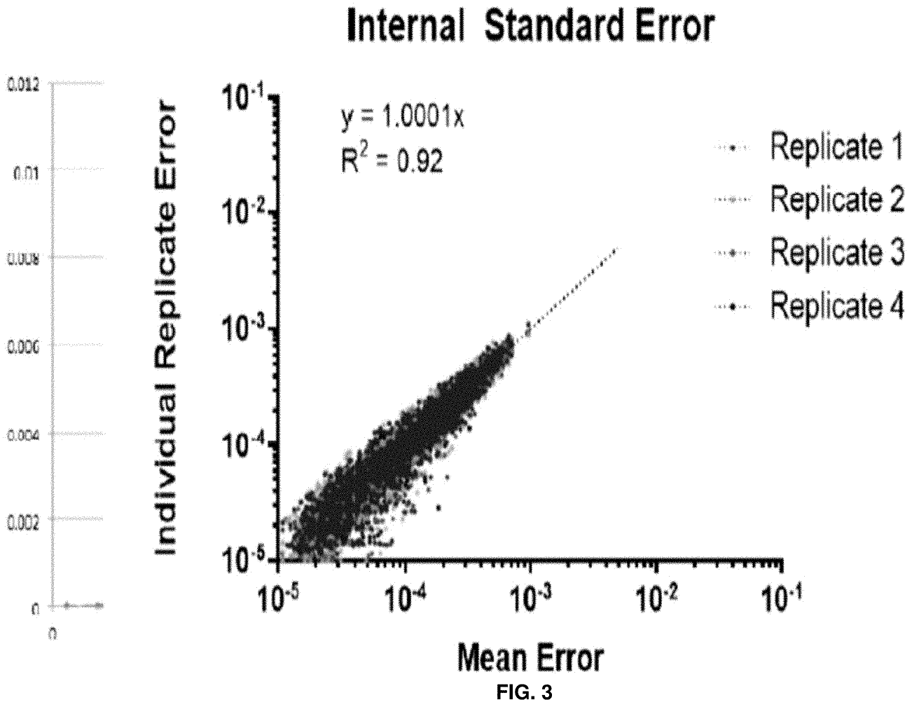

FIGS. 4A-4C show that internal standards enable site-specific LOD (logarithm of the odds). FIG. 4A shows a hybrid capture panel for exons EGFR_18 (red), EGFR_20 (blue) and EGFR_21 (green), showing IS frequency (%). FIG. 4B shows NT frequency (%), showing replicate measurement, LOB, and variant allele frequency for exons EGFR_18 (red), EGFR_20 (blue) and EGFR_21 (green). FIG. 4C shows a comparison of expected, NT, reported NT and reported IS for exons EGFR_18 (red), EGFR_20 (blue) and EGFR_21 (green). Thus, FIGS. 4A-4C show that traditional methods based on external process performance estimates do not support VAF measurements <5%. Also, alternative correction methods are complex and require 10- to 20-fold more sequencing reads.

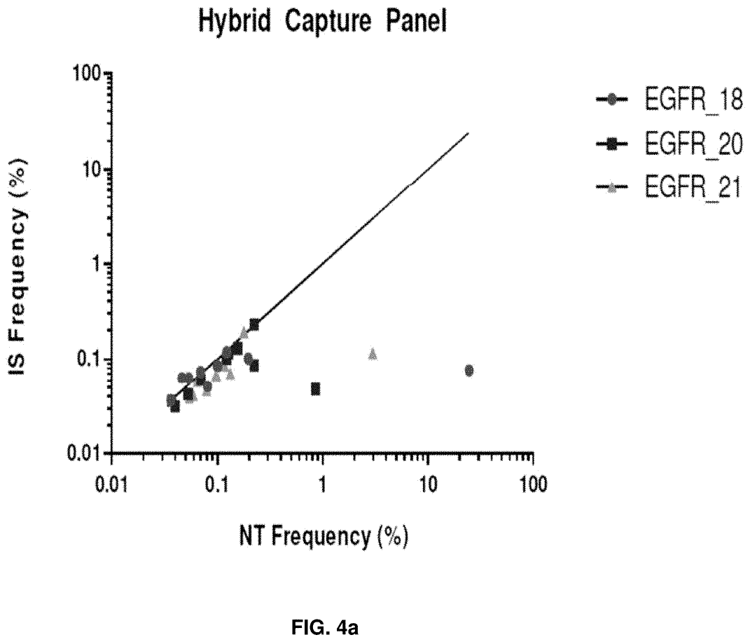

FIG. 5 shows applying Internal Standards (IS) to fragmented FDA samples. The known mutations identified with LOD based on site-specific LOB determined by internal standards (IS).

In one non-limiting example, multiplex gradient PCR enables primers with varying melting temperatures to anneal to specific target. Single-plex PCR followed by quantification and equimolar mixing enables equal loading onto sequencer. PCR targets chosen based on high occurrence in lung cancer and lung premalignant lesions.

Synthetic DNA internal standards (IS) were prepared for each of various lung cancer driver genes and mixed with each AEC genomic (gDNA) specimen prior to competitive multiplex PCR amplicon NGS library preparation. A custom Perl script was developed to separate IS reads and respective specimen gDNA reads from each target into separate files for parallel variant frequency analysis. This approach enabled reliable detection of mutations with VAF as low as 5×10⁻⁴ (0.05%). This method was then applied in a retrospective case-control study. Specifically, AEC specimens were collected by bronchoscopic brush biopsy from the normal airways of 19 subjects, including eleven lung cancer cases and eight non-cancer controls, and the association of lung cancer risk with AEC driver gene mutations was tested.

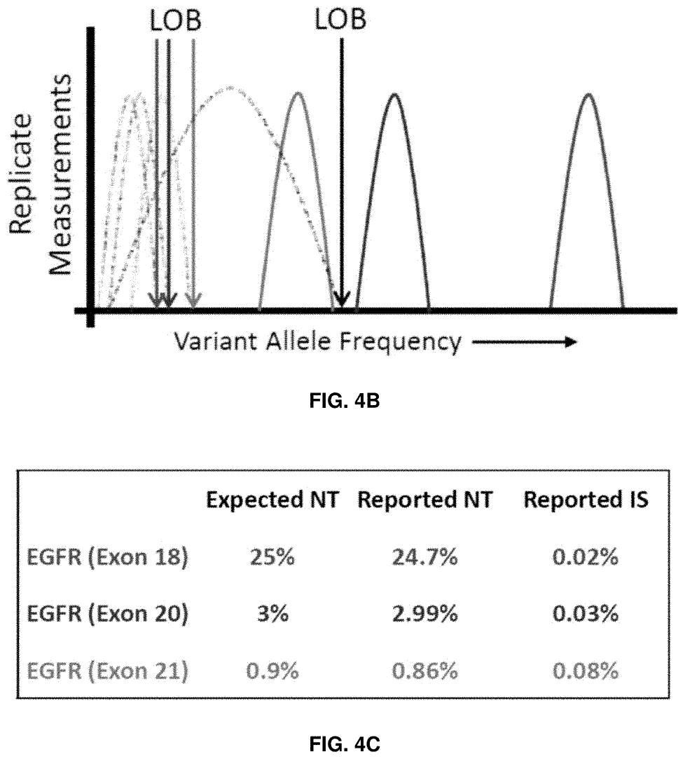

FIG. 6 is an example of transition sequencing error at TP53 (exon 6) across 19 Internal Standard (S) replicates, showing the variant allele frequency (VAF) for TP53 transactivation domain, TP53 DNA binding domain, and TP53 tetramerization domain.

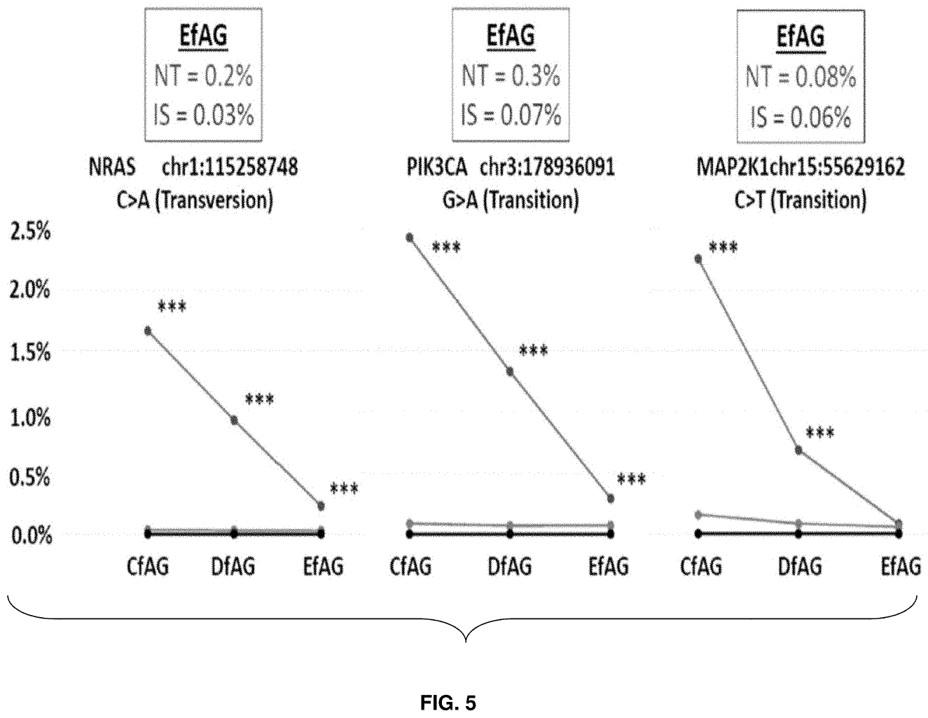

FIG. 7 is an example of transition variants in a sample at TP53 (exon 6), showing the variant allele frequency (VAF) for TP53 transactivation domain, TP53 DNA binding domain, and TP53 tetramerization domain.

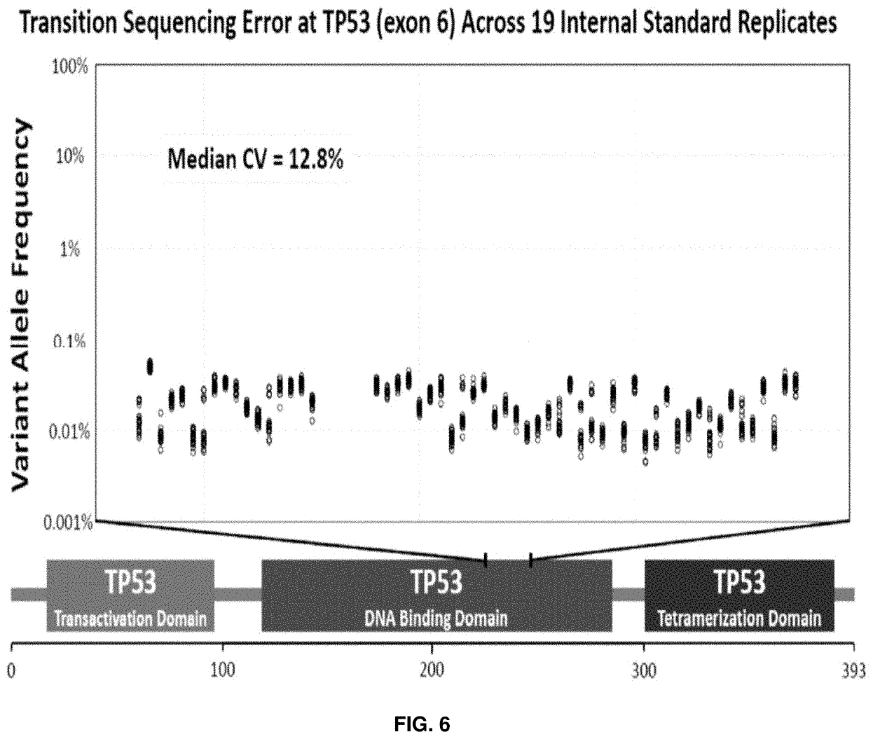

FIG. 8 shows mutations in 19 patient specimens relative to IS. 129 significant variants identified in 19 patient specimens. The VAF for these variants range from 0.05% to 0.46%. 99 variants found in 11 cancer specimens. 30 variants found in 8 non-cancer specimens. Also, there were significant increase in variants of smokers with cancer compared to smokers without cancer.

Also described herein are methods for measurement of low VAF mutants with calculation of limit of detection/limit of quantification for measurement of each analyte in each specimen, based on measurement of specimen analyte relative to a known number of synthetic internal standard molecules.

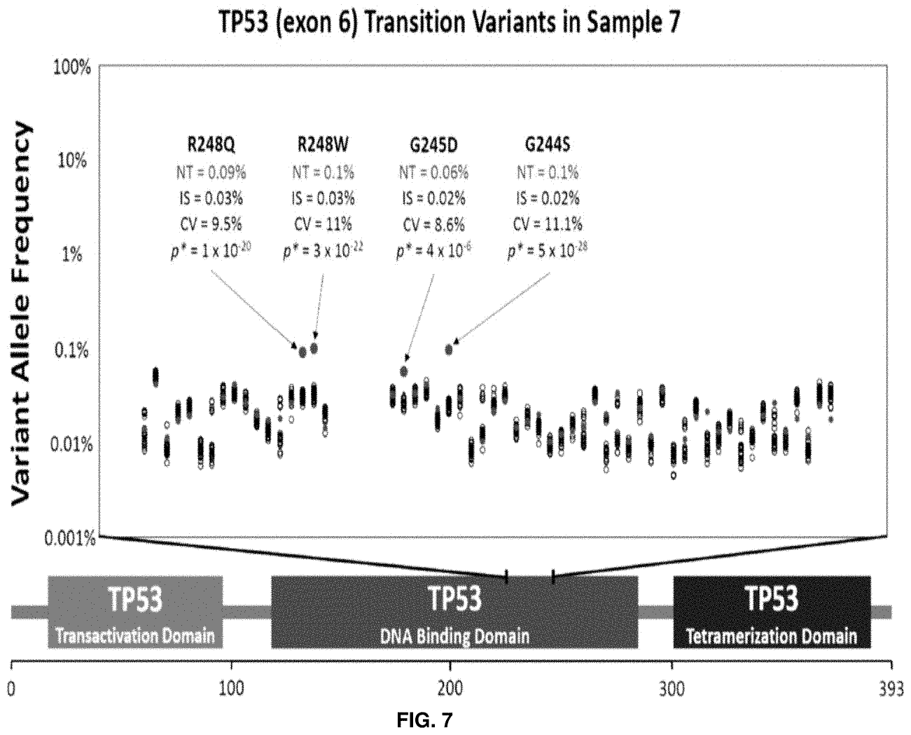

FIG. 9 Example of an endogenous complexity calibration ladder (ECCL). The present method provides a schematic illustration of the method of controlling for NGS library complexity. The method includes providing a mixture of internal standards for a gene target at different concentrations with different nucleotide changes in the internal standard at each of the different concentrations.

In certain embodiments, a kit for practicing this method contains: synthetic nucleic acid internal standard reagents that control for original number of target molecules in a specimen prior to next generation sequencing (NGS) library preparation.

Such kits are useful for NGS molecular diagnostics testing, such as for measurement of variant allele fraction in cancer samples.

Contrary to prior methods and kits, the presently described method and kit simultaneously controls for target copies loaded, technical errors, and type and prevalence of technical errors.

Use of the presently described method and kit increases reliability of NGS clinical diagnostic testing, and reduces sequencing costs.

According to the method, an aliquot of the calibration ladder reagent, containing a known number of genome copies, is loaded into each sample at the prior to library preparation.

An additional option is to combine the “complexity calibration ladder” with an alien sequence ladder that is not competitive with endogenous targets and is not affected by a sample's biological properties. For example, one of the External RNA Controls Consortium (ERCC) sequences can be used as this alien ladder.

Thus, each “complexity calibration ladder” comprises synthetic IS for endogenous (and/or alien) targets and includes IS sequences at different concentrations. Each IS sequence, at each concentration, contains nucleotide changes at different positions along the sequence string—so that a first IS sequence can be distinguished from a second/third/fourth/fifth/etc. IS sequence at each other concentration; and, if applicable, the endogenous sequence at different concentrations, as shown in FIG. 9.

After synthesis of the IS sequences (e.g., first/second/third/fourth/fifth/etc.) with nucleotide changes at different positions, multiple IS with different nucleotide changes are mixed at a different known concentrations relative to each other, and at a known ratio to other target IS in an internal standard mixture.

### EXAMPLES

The methods and embodiments described herein are further defined in the following Examples, in which all parts and percentages are by weight and degrees are Celsius, unless otherwise stated. Certain embodiments of the present invention are defined in the Examples herein. It should be understood that these Examples, while indicating preferred embodiments of the invention, are given by way of illustration only. From the discussion herein and these Examples, one skilled in the art can ascertain the essential characteristics of this invention and without departing from the spirit and scope thereof, can make various changes and modifications of the invention to adapt it to various usages and conditions.

### Example 1

An aliquot of an endogenous complexity calibration ladder (ECCL) reagent, containing a known number of copies of synthetic internal standard molecules, is mixed with each sample prior to library preparation.

In use, each synthetic internal standard (IS) in this ladder will compete with the endogenous target during library preparation, and thereby enable quantification of the number of target copies loaded into the library preparation.

Use of the endogenous complexity calibration ladder (ECCL) of different concentrations of different IS for the target thus enables the quantification of the number of target copies loaded into the library that are captured for loading into the sequencer (also termed library complexity).

The ECCL controls for both sample and library specific variation in complexity.

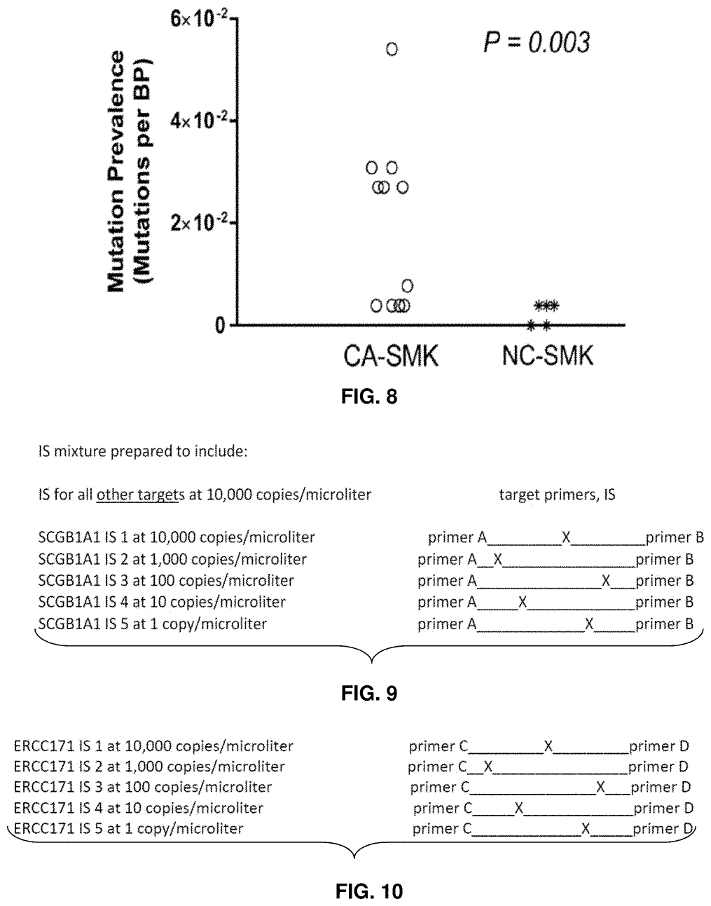

In certain embodiments, there is an option to use an alien complexity calibration ladder (ACCL) that is not competitive with endogenous targets and is not affected by a sample's biological properties. For example, see FIG. 10.

In contrast to the ECCL, the ACCL controls only for library specific variation in complexity. For example, one non-limiting example and ECCL is one of the External RNA Controls Consortium (ERCC) sequences that can serve as the target for this ACCL.

In some embodiments, both the ECCL and ACCL can be mixed with each sample prior to library preparation.

Each ladder (ECCL and ACCL) contain synthetic IS sequences at different concentrations, with the IS at each concentration containing nucleotide changes at different positions along the sequence string so that they can be distinguished from the IS at each other concentration and, if applicable, the endogenous sequence at different concentrations.

After synthesis of IS with nucleotide changes at different positions, multiple IS with different nucleotide changes are mixed at different known concentrations relative to each other and at a known ratio to IS for other targets in an internal standard mixture.

In certain embodiments, for other targets, a single IS concentration is used. However, in other certain embodiments, there may be conditions for which ACCL for multiple targets are used.

### Example 2

IS mixture prepared to include IS for all other targets at a single concentration of 10,000 copies/microliter; and, ECCL comprising IS for an endogenous target (in this case SCGB1A1) as can be seen by combining the information shown in FIG. 9 with the specimen.

IS/specimen target genome copy ratio has a goal of 1:1.

Then, the IS/specimen mixture subjected to usual library preparation.

The sequencing includes where the IS/specimen library preparation subjected to a standard protocol.

### Example 3

A sequencing according to Example 2, an analysis (called Complexity Analysis herein) is conduced. The Complexity Analysis involves the analysis of sequencing reads to determine the complexity of the library preparation for each target in each specimen.

The Complexity Analysis compares the reads of each other target IS to each IS in complexity ladder to determine efficiency of library preparation for each target in each specimen, and, therefore, the number of molecules measured for each target in each specimen.

For ECCL, the Complexity Analysis includes the following steps:

1) identifying ECCL IS minD=copies of ECCL IS loaded for which there are at least 5 reads (or some chosen minimum reads);

2) determining ECCL detection correction factor=ECCL IS minL/ECCL IS minD where ECCL IS minL is the number of copies loaded of the least concentrated IS in the ECCL;

3) calculating ECCL IS1 detected by multiplying IS1 copies loaded by the ECCL detection correction factor;

4) calculating ECCL NT copies detected using the formula [(ECCL NT reads/ECCL IS1 reads)*ECCL IS1 copies detected; and,

5) calculating detectable target NT copies using the formula [(target NT reads/ECCL NT reads)*ECCL NT copies detected*(target IS loaded/ECCL IS1 loaded).

In certain embodiments, there is an option of calculating the ECCL NT copies detected against each ECCL and/or ACCL IS and using the average or median to calculate detectable target NT copies.

For ACCL, the Complexity Analysis includes the following steps:

1) identifying ACCL IS minD=copies of ACCL IS loaded for which there are at least 5 reads (or some chosen minimum reads);

2) determining ACCL detection correction factor=ACCL IS minL/ACCL IS minD where ACCL IS minL is the number of copies loaded of the least concentrated IS in the ACCL;

3) calculating ACCL IS1 detected by multiplying IS1 copies loaded by the ACCL detection correction factor;

4) calculating target IS copies detected by multiplying ACCL IS1 copies detected by target IS reads/ACCL IS1 read; and,

5) calculating target NT copies detected by multiplying target IS copies detected by target NT reads/target IS reads.

Non-Limiting Examples of Applications

The example ratios in FIGS. 8 and 9 are not limiting. That is, additional examples can include a finer titration such as 1 molecule, 2 molecules, 3 molecules . . . 10 molecules or 1, 3, 9 . . . 100 molecules, etc.). Thus, any ratio can be used in the ladder and any number of different oligonucleotides can be included in a ladder.

In some embodiments, a method for obtaining a numerical index that indicates a biological state comprises providing 2 samples corresponding to each of a first biological state and a second biological state; measuring and/or enumerating an amount of each of 2 nucleic acids in each of the 2 samples; providing the amounts as numerical values that are directly comparable between a number of samples; mathematically computing the numerical values corresponding to each of the first and second biological states; and determining a mathematical computation that discriminates the two biological states. First and second biological states as used herein correspond to two biological states of to be compared, such as two phenotypic states to be distinguished. Non-limiting examples include, e.g., non-disease (normal) tissue vs. disease tissue; a culture showing a therapeutic drug response vs. a culture showing less of the therapeutic drug response; a subject showing an adverse drug response vs. a subject showing a less adverse response; a treated group of subjects vs. a non-treated group of subjects, etc.

A “biological state” as used herein can refer to a phenotypic state, for e.g., a clinically relevant phenotype or other metabolic condition of interest. Biological states can include, e.g., a disease phenotype, a predisposition to a disease state or a non-disease state; a therapeutic drug response or predisposition to such a response, an adverse drug response (e.g. drug toxicity) or a predisposition to such a response, a resistance to a drug, or a predisposition to showing such a resistance, etc. In preferred embodiments, the numerical index obtained can act as a biomarker, e.g., by correlating with a phenotype of interest. hi some embodiments, the drug may be and anti-tumor drug. In certain embodiments, the use of the method described herein can provide personalized medicine.

In certain embodiments, the biological state corresponds to a normal expression level of a gene. Where the biological state does not correspond to normal levels, for example falling outside of a desired range, a non-normal, e.g., disease condition may be indicated.

A numerical index that discriminates a particular biological state, e.g., a disease or metabolic condition, can be used as a biomarker for the given condition and/or conditions related thereto. For example, in some embodiments, the biological state indicated can be at least one of an angiogenesis-related condition, an antioxidant-related condition, an apoptosis-related condition, a cardiovascular-related condition, a cell cycle-related condition, a cell structure-related condition, a cytokine-related condition, a defense response-related condition, a development-related condition, a diabetes-related condition, a differentiation-related condition, a DNA replication and/or repair-related condition, an endothelial cell-related condition, a hormone receptor-related condition, a folate receptor-related condition, an inflammation-related condition, an intermediary metabolism-related condition, a membrane transport-related condition, a neurotransmission-related condition, a cancer-related condition, an oxidative metabolism-related condition, a protein maturation-related condition, a signal transduction-related condition, a stress response-related condition, a tissue structure-related condition, a transcription factor-related condition, a transport-related condition, and a xenobiotic metabolism-related condition. In other specific embodiments, antioxidant and xenobiotic metabolism enzyme genes can be evaluated in human cells; micro-vascular endothelial cell gene expression; membrane transport genes expression; immune resistance; transcription control of hormone receptor expression; and gene expression patterns with drug resistance in carcinomas and tumors.

In some embodiments, one or more of the nucleic acids to be measured are associated with one of the biological states to a greater degree than the other(s). For example, in some embodiments, one or more of the nucleic acids to be evaluated is associated with a first biological state and not with a second biological state.

A nucleic acid may be said to be “associated with” a particular biological state where the nucleic acid is either positively or negatively associated with the biological state. For example, a nucleic acid may be said to be “positively associated” with a first biological state where the nucleic acid occurs in higher amounts in a first biological state compared to a second biological state. As an illustration, genes highly expressed in cancer cells compared to non-cancer cells can be said to be positively associated with cancer. On the other hand, a nucleic acid present in lower amounts in a first biological state compared to a second biological state can be said to be negatively associated with the first biological state.

The nucleic acid to be measured and/or enumerated may correspond to a gene associated with a particular phenotype. The sequence of the nucleic acid may correspond to the transcribed, expressed, and/or regulatory regions of the gene (e.g., a regulatory region of a transcription factor, e.g., a transcription factor for co-regulation).

In some embodiments, expressed amounts of more than 2 genes are measured and used in to provide a numerical index indicative of a biological state. For example, in some cases, expression patterns of multiple genes are used to characterize a given phenotypic state, e.g., a clinically relevant phenotype. In some embodiments, expressed amounts of at least about 5 genes, at least about 10 genes, at least about 20 genes, at least about 50 genes, or at least about 70 genes may be measured and used to provide a numerical index indicative of a biological state. In some embodiments of the instant invention, expressed amounts of less than about 90 genes, less than about 100 genes, less than about 120 genes, less than about 150 genes, or less than about 200 genes may be measured and used to provide a numerical index indicative of a biological state.

Determining which mathematic computation to use to provide a numerical index indicative of a biological state may be achieved by any methods known in the arts, e.g., in the mathematical, statistical, and/or computational arts. In some embodiments, determining the mathematical computation involves a use of software. For example, in some embodiments, a machine learning software can be used.

Mathematically computing numerical values can refer to using any equation, operation, formula and/or rule for interacting numerical values, e.g., a sum, difference, product, quotient, log power and/or other mathematical computation. In some embodiments, a numerical index is calculated by dividing a numerator by a denominator, where the numerator corresponds to an amount of one nucleic acid and the denominator corresponds to an amount the another nucleic acid. In certain embodiments, the numerator corresponds to a gene positively associated with a given biological state and the denominator corresponds to a gene negatively associated with the biological state. In some embodiments, more than one gene positively associated with the biological state being evaluated and more than one gene negatively associated with the biological state being evaluated can be used. For example, in some embodiments, a numerical index can be derived comprising numerical values for the positively associated genes in the numerator and numerical values for an equivalent number of the negatively associated genes in the denominator. In such balanced numerical indices, the reference nucleic acid numerical values cancel out. In some embodiments, balanced numerical values can neutralize effects of variation in the expression of the gene(s) providing the reference nucleic acid(s). In some embodiments, a numerical index is calculated by a series of one or more mathematical functions.

In some embodiments, more than 2 biological states can be compared, e.g., distinguished. For example, in some embodiments, samples may be provided from a range of biological states, e.g., corresponding to different stages of disease progression, e.g., different stages of cancer. Cells in different stages of cancer, for example, include a non-cancerous cell vs. a non-metastasizing cancerous cell vs. a metastasizing cell from a given patient at various times over the disease course. In preferred embodiments, biomarkers can be developed to predict which chemotherapeutic agent can work best for a given type of cancer, e.g., in a particular patient.

A non-cancerous cell may include a cell of hematoma and/or scar tissue, as well as morphologically normal parenchyma from non-cancer patients, e.g., non-cancer patients related or not related to a cancer patient. Non-cancerous cells may also include morphologically normal parenchyma from cancer patients, e.g., from a site close to the site of the cancer in the same tissue and/or same organ; from a site further away from the site of the cancer, e.g., in a different tissue and/or organ in the same organ-system, or from a site still further away e.g., in a different organ and/or a different organ-system.

Numerical indices obtained can be provided as a database. Numerical indices and/or databases thereof can find use in diagnoses, e.g. in the development and application of clinical tests.

Diagnostic Applications

In some embodiments, a method of identifying a biological state is provided. In some embodiments, the method comprises measuring and/or enumerating an amount of each of 2 nucleic acids in a sample, providing the amounts as numerical values; and using the numerical values to provide a numerical index, whereby the numerical index indicates the biological state.

A numerical index that indicates a biological state can be determined as described above in accordance with various embodiments. The sample may be obtained from a specimen, e.g., a specimen collected from a subject to be treated. The subject may be in a clinical setting, including, e.g., a hospital, office of a health care provider, clinic, and/or other health care and/or research facility. Amounts of nucleic acid(s) of interests in the sample can then be measured and/or enumerated.

In certain embodiments, where a given number of genes are to be evaluated, expression data for that given number of genes can be obtained simultaneously. By comparing the expression pattern of certain genes to those in a database, a chemotherapeutic agent that a tumor with that gene expression pattern would most likely respond to can be determined.

In some embodiments, the methods can be used to quantify exogenous normal gene in the presence of mutated endogenous gene. Using primers that span the deleted region, one can selectively amplify and quantitate expression from a transfected normal gene and/or a constitutive abnormal gene.

In some embodiments, methods described herein can be used to determine normal expression levels, e.g., providing numerical values corresponding to normal gene transcript expression levels. Such embodiments may be used to indicate a normal biological state, at least with respect to expression of the evaluated gene.

Normal expression levels can refer to the expression level of a transcript under conditions not normally associated with a disease, trauma, and/or other cellular insult. In some embodiments, normal expression levels may be provided as a number, or preferably as a range of numerical values corresponding to a range of normal expression of a particular gene, e.g., within +/− a percentage for experimental error. Comparison of a numerical value obtained for a given nucleic acid in a sample, e.g., a nucleic acid corresponding to a particular gene, can be compared to established-normal numerical values, e.g., by comparison to data in a database provided herein. As numerical values can indicate numbers of molecules of the nucleic acid in the sample, this comparison can indicate whether the gene is being expressed within normal levels or not.

In some embodiments, the method can be used for identifying a biological state comprising assessing an amount a nucleic acid in a first sample, and providing said amount as a numerical value wherein said numerical value is directly comparable between a number of other samples. In some embodiments, the numerical value is potentially directly comparable to an unlimited number of other samples. Samples may be evaluated at different times, e.g., on different days; in the same or different experiments in the same laboratory; and/or in different experiments in different laboratories.

Therapeutic Applications

Some embodiments provide a method of improving drug development. For example, use of a standardized mixture of internal standards, a database of numerical values and/or a database of numerical indices may be used to improve drug development.

In some embodiments, modulation of gene expression is measured and/or enumerated at one or more of these stages, e.g., to determine effect a candidate drug. For example, a candidate drug (e.g., identified at a given stage) can be administered to a biological entity. The biological entity can be any entity capable of harboring a nucleic acid, as described above, and can be selected appropriately based on the stage of drug development. For example, at the lead identification stage, the biological entity may be an in vitro culture. At the stage of a clinical trial, the biological entity can be a human patient.

The effect of the candidate drug on gene expression may then be evaluated, e.g., using various embodiments of the instant invention. For example, a nucleic acid sample may be collected from the biological entity and amounts of nucleic acids of interest can be measured and/or enumerated. For example, amounts can be provided as numerical value and/or numerical indices. An amount then may be compared to another amount of that nucleic acid at a different stage of drug development; and/or to a numerical values and/or indices in a database. This comparison can provide information for altering the drug development process in one or more ways.

Altering a step of drug development may refer to making one or more changes in the process of developing a drug, preferably so as to reduce the time and/or expense for drug development. For example, altering may comprise stratifying a clinical trial. Stratification of a clinical trial can refer to, e.g., segmenting a patient population within a clinical trial and/or determining whether or not a particular individual may enter into the clinical trial and/or continue to a subsequent phase of the clinical trial. For example, patients may be segmented based on one or more features of their genetic makeup determined using various embodiments of the instant invention. For example, consider a numerical value obtained at a pre-clinical stage, e.g., from an in vitro culture that is found to correspond to a lack of a response to a candidate drug. At the clinical trial stage, subjects showing the same or similar numerical value can be exempted from participation in the trial. The drug development process has accordingly be altered, saving time, and costs.

Kits

The internal standards (IS) described herein may be assembled and provided in the form of kits. In some embodiments, the kit provides the reagents necessary to perform a PCR, including Multiplex-PCR and next-generation sequencing (NGS).

Also, in certain embodiments, the kits also may contain oligonucleotide “baits” to capture IS and/or NT sequence fragments. Baits are oligonucleotides that retrieve specific RNA species or genomic DNA fragments of interest for sequencing. The desired DNA or RNA molecules hybridize with the baits, and others do not.

The kits may include IS of multiple identified endogenous targets, as described herein, and/or IS of various alien targets, as described herein, or both.

These IS may be provided in solution allowing the IS to remain stable for up to several years.

The kits may also provide primers designed specifically to amplify the IS of the endogenous targets, the IS of alien targets, and their corresponding native targets.

The kits may also provide one or more containers filled with one or more necessary PCR reagents, including but not limited to dNTPs, reaction buffer, Taq polymerase, and RNAse-free water. Optionally associated with such container(s) is a notice in the form prescribed by a governmental agency regulating the manufacture, use or sale of IAC and associated reagents, which notice reflects approval by the agency of manufacture, use or sale for research use.

The kits may include appropriate instructions for preparing, executing, and analyzing PCR, including Multiplex-PCR and NGS, using the IS included in the kit. The instructions may be in any suitable format, including, but not limited to, printed matter, videotape, computer readable disk, or optical disc.

All publications, including patents and non-patent literature, referred to in this specification are expressly incorporated by reference herein. Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

What is claimed is:

1. A kit for quantifying the amount of at least one nucleic acid of interest in a sample, comprising:
spike-in internal standard (IS) reagents present as a complexity calibration ladder (CCL) that contain multiple synthetic internal standard (IS) sequences at different concentrations,
wherein the IS sequence, at each concentration, contains a nucleotide change at a different position along the sequence so that each IS sequence is distinguishable from the IS sequence at each other concentration; and
wherein the multiple internal standard (IS) sequences are mixed at different known concentrations relative to each other, and at a known ratio to IS for other targets in an internal standard

2. The kit of claim 1, wherein the spike-in IS reagents comprise one or more of:
i) an endogenous complexity calibration ladder (ECCL) that includes synthetic internal standard competitors for at least one endogenous target gene; and,
ii) an alien complexity calibration ladder (ACCL) that includes synthetic internal standard competitors for at least one alien target gene.

3. The kit or claim 1, wherein the endogenous complexity calibration ladder (ECCL) is combined with an alien complexity calibration ladder (ACCL) that is not competitive with the at least one endogenous target gene and is not affected by a sample's biological properties.

4. The kit of claim 1, wherein the alien complexity calibration ladder (ACCL) comprises at least one of the External RNA Controls Consortium (ERCC) sequences.

5. The kit of claim 1, wherein the endogenous complexity calibration ladder (ECCL) and/or the alien complexity calibration ladder (ACCL) comprises synthetic IS for the endogenous and/or alien target and includes IS sequences at different concentrations.

6. The kit of claim 1, further comprising reagents for measurement of expression and/or somatic mutations in multiple genes in a sample of cells, the kit including:
a) PCR primers for each target gene;
b) synthetic internal standard for each target gene; and,
c) reagents to prepare PCR products as a library for next generation sequencing, and/or oligonucleotides baits to capture IS and/or NT sequence fragments.

7. The kit of claim 1, wherein the nucleic acid of interest comprises one or more of:
RNA to be measured, mRNA, DNA, cDNA, genomic DNA and variant alleles

8. A method using the kit of claim 1, for quantifying the amount of at least one nucleic acid of interest in a sample, the method comprising:
a) comparing the reads of each other target IS to each IS in a complexity ladder, and
b) determining the efficiency of library preparation for each target in each sample, and,
c) determining the number of molecules measured for each target in each sample.

9. The method of claim 8, wherein the complexity analysis of step a) includes the following steps:
1) identifying ECCL IS minD=copies of ECCL IS loaded for which there are at least 5 reads (or some chosen minimum reads);
2) determining ECCL detection correction factor=ECCL IS minL/ECCL IS minD where ECCL IS minL is the number of copies loaded of the least concentrated IS in the ECCL;
3) calculating ECCL IS1 detected by multiplying IS1 copies loaded by the ECCL detection correction factor;
4) calculating ECCL NT copies detected using the formula [(ECCL NT reads/ECCL IS1 reads)*ECCL IS1 copies detected; and,
5)calculating detectable target NT copies using the formula [(target NT reads/ECCL NT reads)*ECCL NT copies detected*(target IS loaded/ECCL IS1 loaded).

10. The method of claim 8, wherein the complexity analysis of step a) includes the following steps:
1) identifying ACCL IS minD=copies of ACCL IS loaded for which there are at least 5 reads (or some chosen minimum reads);
2) determining ACCL detection correction factor=ACCL IS minL/ACCL IS minD where ACCL IS minL is the number of copies loaded of the least concentrated IS in the ACCL;
3) calculating ACCL IS1 detected by multiplying IS1 copies loaded by the ACCL detection correction factor;
4) calculating target IS copies detected by multiplying ACCL IS1 copies detected by target IS reads/ACCL IS1 read; and,
5) calculating target NT copies detected by multiplying target IS copies detected by target NT reads/target IS reads.

11. The method of claim 9, further including the step of: 6) calculating the ECCL NT copies detected against each ECCL IS and using the average or median to calculate detectable target NT copies.

12. The method of claim 8, wherein inclusion of the internal standards (IS) measures mutations at a variant frequency as low as 0.05%, and 5% without the inclusion of the internal standards (IS)

13. The method of claim 8, wherein the use of the internal standards (IS) measures low variant frequency mutations with variant allele frequency (VAF) as low as 0.01% without use of unique molecular indices (UMI).

14. The method of claim 8, comprising using the kit for diagnosis or evaluation of one or more of: a diagnosis of a disease, a diagnosis of a stage of the disease, a diagnosis of a type or classification of the disease, a diagnosis or detection of a recurrence of the disease, a diagnosis or detection of a regression of the disease, a prognosis of the disease, and an evaluation of the response of a disease to a surgical or non-surgical therapy.

15. The method of claim 14, further comprising using the kit to facilitate approval by FDA and other regulatory agencies of testing for measurement without unique molecular indices (UMI) of very low VAF mutations in cells, and guiding targeted therapy of the disease in kit or method form in regional laboratories.
